SEQUENCE LISTING (28 sequences)

SEQ ID NO: 1 - DNA, 23 bp, Mus musculus
cctgaagctccaagaataccaaa

SEQ ID NO: 2 - DNA, 28 bp, Mus musculus
agagttgggttttttcagaataataagg

SEQ ID NO: 3 - DNA, 20 bp, Mus musculus
tcgattcacctacagcacgc

SEQ ID NO: 4 - DNA, 21 bp, Mus musculus
gactgtcttgccccaagttcc

SEQ ID NO: 5 - DNA, 20 bp, Mus musculus
ttcccactggctgaaaaggt

SEQ ID NO: 6 - DNA, 15 bp, Mus musculus
cgcagccgcaaatgc

SEQ ID NO: 7 - DNA, 18 bp, Mus musculus
agtggagtggtgctgaag

SEQ ID NO: 8 - DNA, 18 bp, Mus musculus
gccgtcctttttcagttg

SEQ ID NO: 9 - DNA, 19 bp, Mus musculus
agaagtcacccgcgtgcta

SEQ ID NO: 10 - DNA, 24 bp, Mus musculus
tgtgtgatgtctttggttttgtca

SEQ ID NO: 11 - DNA, 18 bp, Mus musculus
gtgaagaagccgaaaatg

SEQ ID NO: 12 - DNA, 18 bp, Mus musculus
aatgttttccttttgatg

SEQ ID NO: 13 - DNA, 18 bp, Mus musculus
atcctataacaatgcagt

SEQ ID NO: 14 - DNA, 18 bp, Mus musculus
ttcatctctgtgcccttc

SEQ ID NO: 15 - DNA, 20 bp, Mus musculus
tgtgctggactctggagatg

SEQ ID NO: 16 - DNA, 18 bp, Mus musculus
gatcacctgcccatcagg

SEQ ID NO: 17 - DNA, 19 bp, Mus musculus
cgagaatgcggatccacaa

SEQ ID NO: 18 - DNA, 20 bp, Mus musculus
ccgcttcaggttttccgtta

SEQ ID NO: 19 - DNA, 21 bp, Mus musculus
catcgccagtcacatgcacct

SEQ ID NO: 20 - DNA, 21 bp, Mus musculus
cgtctatgccatgcgagctca

SEQ ID NO: 21 - DNA, 18 bp, Mus musculus
caggacctgctcaatgtg

SEQ ID NO: 22 - DNA, 23 bp, Mus musculus
gtagcctcgctgctgacaacctc

SEQ ID NO: 23 - DNA, 20 bp, Mus musculus
ctgcccgtagacaaaatggt

SEQ ID NO: 24 - DNA, 20 bp, Mus musculus
gaatttgccgtgagtggagt

SEQ ID NO: 25 - DNA, 20 bp, Mus musculus
ctgagcgtggctattccttc

SEQ ID NO: 26 - DNA, 20 bp, Mus musculus
cctctgcatcctgtcagcaa

SEQ ID NO: 27 - DNA, 21 bp, Mus musculus
cagtaaggcctgtagctgtgc

SEQ ID NO: 28 - DNA, 19 bp, Mus musculus
ctcgttgatttcggggaac

(19) United States
(12) Patent Application Publication
(10) Pub. No.: US 2016/0106770 A1
(43) Pub. Date: Apr. 21, 2016

(54) EPIGENETIC MECHANISMS OF ANTI-FIBROTIC ACTION FOR THE LIVER

(71) Applicant: University of Southern California, Los Angeles, CA (US)

(72) Inventors: Hidekazu Tsukamoto, La Canada Flintridge, CA (US); Clay Wang, San Marino, CA (US)

(73) Assignee: University of Southern California, Los Angeles, CA (US)

(21) Appl. No.: 14/987,365

(22) Filed: Jan. 4, 2016

Related U.S. Application Data
(63) Continuation of application No. 13/691,604, filed on Nov. 30, 2012.
(60) Provisional application No. 61/565,275, filed on Nov. 30, 2011.

(51) Int. Cl. A61K 31/7048

(52) U.S. Cl. CPC A61K 31/7048

(57) ABSTRACT

This invention demonstrates the therapeutic efficacy of Yang-Gan-Wan (YGW) and its active components, especially in the formulation provided by Sheng-Pu Pharmaceutials, Inc., for treating and preventing liver fibrosis. This invention further demonstrates MeCP2 is an important therapeutic target for YGW and its active ingredients' action against liver fibrosis and cirrhosis.

### CROSS-REFERENCE TO RELATED APPLICATION

This application claims priority from U.S. Provisional Patent Application Number 61/565,275, filed on Nov. 30, 2011, which is incorporated herein by reference in its entirety.

### STATEMENT REGARDING FEDERALLY SPONSORED RESEARCH

The U.S. Government has a paid-up license in this invention and the right in limited circumstances to require the patent owner to license others on reasonable terms as provided for by the terms of Grant Nos.: R37AA06603, P5OAA011999, and R24AA12885, awarded by the NIH.

### FIELD OF THE INVENTION

The present invention generally relates to the prevention and treatment of liver fibrosis.

### SUMMARY OF THE INVENTION

In some embodiments, the invention teaches a method for treating and/or inhibiting liver fibrosis in a subject, including providing a therapeutically effective amount of a composition that inhibits or reduces epigenetic repression of Pparγ to the subject. In certain embodiments, the composition includes two or more plants selected from the group consisting of: Angelica Sinensis, Paeoniae Albiflora, Radix Rehmannae Preparate, and Ligustici Wallichii Rhizoma. In some embodiments, the composition includes Yang-Gan-Wan. In some embodiments, the composition includes rosmarinic acid. In some embodiments, the composition includes baicalin. In some embodiments, the composition reduces the level of MeCP2 expression in the subject. In certain embodiments, the composition reduces or eliminates activation of a hepatic stellate cell (HSC) in the subject and/or leads to a quiescent state in said cell.

In various embodiments, the invention teaches a composition for treating and/or inhibiting liver fibrosis in a subject. In some embodiments, the composition includes two or more plants selected from the group consisting of: Angelica Sinensis, Paeoniae Albiflora, Radix Rehmannae Preparate, and Ligustici Wallichii Rhizoma. In some embodiments, the composition includes rosmarinic acid. In some embodiments, the composition includes baicalin. In some embodiments, the composition reduces repression of Pparγ when administered to the subject. In certain embodiments, the composition reduces a level of MeCP2 in the subject when administered. In certain embodiments, the composition reduces, eliminates or reverses activation of a hepatic stellate cell in the subject and/or leads to a quiescent state in said cell, when administered to the subject.

In various embodiments, the invention teaches a kit for treating and/or inhibiting liver fibrosis in a subject. In some embodiments, the kit includes a composition including two or more plants selected from the group consisting of: Angelica Sinensis, Paeoniae Albiflora, Radix Rehmannae Preparate, and Ligustici Wallichii Rhizoma; and instructions for the use thereof to treat and/or inhibit liver fibrosis in the subject. In certain embodiments, the composition reduces repression of Pparγ in the subject when a therapeutically effective dose is administered. In certain embodiments, the kit includes Yang-Gan-Wan. In certain embodiments, the kit includes rosmarinic acid. In some embodiments, the kit includes baicalin. In some embodiments, the composition reduces the level of MeCP2 in the subject, when a therapeutically effective dose is administered. In some embodiments, the composition reduces activation of a hepatic stellate cell in the subject and/or leads to a quiescent state of said cell, when a therapeutically effective dose is administered.

### BACKGROUND

The following description includes information that may be useful in understanding the present invention. It is not an admission that any of the information provided herein is prior art or relevant to the presently claimed invention.

Excessive scarring of the liver results in cirrhosis, the end-stage liver disease of high mortality for which efficacious medical treatments are not currently available, except for liver transplantation. Thus, there is a need in the art for therapeutic strategies for liver fibrosis.

### BRIEF DESCRIPTION OF THE DRAWINGS

Exemplary embodiments are illustrated in the referenced figures. It is intended that the embodiments and figures disclosed herein are to be considered illustrative rather than restrictive.

FIG. 1 demonstrates, in accordance with an embodiment of the invention, Yang-Gan Wan (YGW) prevents and reverses hepatic stellate cell (HSC) activation in culture. A. Phase contrast microscopy of activating day 3 or fully-activated day 7 rat HSCs cultured for the last 48 hrs with YGW extract, vehicle control, or no addition. A morphologic reversal of activated HSCs to quiescent cells can be seen. B. Immunostaining for SMA. A marked reduction in SMA with YGW extract can be seen. C. Oil red O staining after retinol and palmitate addition is increased in YGW treated 7 day HSCs. D. The mRNA levels for activation marker genes, α1(I)collagen, αSMA, and TGF-β1 are conspicuously suppressed in day 7 HSCs by the 48-hr treatment with YGW extract, while PPARγ mRNA is induced. *p<0.05, **p<0.01 compared to the vehicle control treatment.

FIG. 2 demonstrates, in accordance with an embodiment of the invention, PPARγ epigenetic repression is lifted with YGW extract. A. Recruitment of Ser2-p RNA polymerase II to the Pparγ gene is significantly reduced in day 7 culture-activated HSCs with no addition or with the vehicle control treatment, and this reduction is attenuated by the YGW extract treatment. B. Increased MeCP2 recruitment to the Pparγ promoter in day 7 culture-activated HSCs is normalized with the YGW extract. C. MeCP2 protein detected by immunoblotting in day 5 HSCs cultured for 24 and 48 hr with the vehicle control becomes undetectable by the YGW treatment. D. Increased H3K27me2 at the Pparγ exon 2 locus in day 7 HSCs is reduced with the YGW extract. E. Increased mRNA levels of the PRC2 component EZH2, Suz12, and EED in day 7 HSCs are reduced by the YGW treatment. F. H3K4me2 at the Pparγ promoter locus is increased by the YGW extract treatment in day 7 HSCs compared to HSCs treated with the vehicle. G. Reduced H3 acetylation (H3Ac) in 7 day HSCs is attenuated with the YGW extract. *p<0.05 compared to day 1 HSCs, †p<0.05 compared to the vehicle control.

FIG. 3 demonstrates, in accordance with an embodiment of the invention, suppression of IKK and NF-κB with YGW. A. Day 5 HSCs cultured with the YGW extract vs. the vehicle control for 6 or 24 hr in serum-free medium, show reduced IKK activity as assessed by phosphorylation of IκBα-GST fusion protein. A positive control for IKK activation is shown with LPS-stimulated RAW macrophage cell line (last lane). B. Day 5 HSCs cultured with the YGW extract for 24 or 48 hr, show marked reductions in the levels of IκBα and IκBβ proteins, as well as in type I collagen protein. C. The activity of κB promoter is significantly reduced by the YGW extract in the rat HSC line (BSC) as assessed by a transient transfection reporter analysis. *p<0.05.

FIG. 4 demonstrates, in accordance with an embodiment of the invention, identification of active components. A. Treatment of day 7 HSCs with a gel filtration fraction with a molecular mass range of 200˜750 Da, causes a morphologic reversal of HSCs as compared to the cells treated with the elution buffer control (phase contract microscopy). B and C. Addition of the fraction to 7 day HSC culture reduces increased α1(I) collagen mRNA and MeCP2 enrichment to the Pparγ promoter as shown with the YGW extract. D. A summary of chromatographic methods for separation of YGW's active ingredients. E. Butanol (BuOH) fraction A and B eluted with 10% acetonitrile-90% water and 40% acetonitrile-60% water, respectively, produce reproducible effects of HSC morphologic reversal as shown by phase contrast microscopy and oil red O staining. F. LC/MS tracing of butanol fractions identifies 5 peaks of which two are identified to be RA and BC. G. Molecular structures of rosmarinic acid and baicalin.

FIG. 5 demonstrates, in accordance with an embodiment of the invention, rosmarinic acid (RA) and baicalin (BC) are active components of YGW that render epigenetic de-repression of Pparγ. A. Both rosmarinic acid (RA) and baicalin (BC) reverse activated HSC to quiescent cells as shown by phase contrast and UV-excited autofluorescence microscopy. B. RA and BC (270 μM) reduce mRNA expression for α1(I) collagen and increase that for PPARγ. C. RA and BC reduce MeCP2 protein level in day 7 HSCs. D. MeCP2 enrichment to the Pparγ promoter is reduced with RA and BC. E. EZH2 mRNA level is reduced equally by RA and BC. F. H3K27me2 at the Pparγ exon 2 is reduced by RA and BC. *p<0.05 compared to the solvent control. G. RA and BC (270 μM) reduce the expression of Wnt10b, Wnt3a, and Necdin in day 7 HSCs compared to the vehicle control as determined by qPCR analysis. H. RA (shaded bar) and BC (black bar) reduce the TOPFLASH promoter activity in day 7 primary HSCs as determined by a transient transfection using an electroporation method. I. RA treatment (ip injection daily at 0.1 mg/25 g body weight) given during the last week of the two-week cholestasis caused by the bile duct ligation, attenuates liver fibrosis in mice as assessed by digital morphometric analysis of Sirius red staining. *p<0.05 compared to the vehicle control. J. Hepatic expression of α1(I) procollagen and SMA are also significantly reduced by the RA treatment. *p<0.05 compared to Sham. +p<0.05 compared to vehicle-treated (Cont) mice.

FIG. 6 demonstrates, in accordance with an embodiment of the invention, prolonged YGW treatment causes HSC apoptosis in culture. Rat primary HSCs cultured on plastic for 5 days, were treated with the YGW extract or vehicle for 8 days with replenishment of the medium with the extract every 2 days. The cells were then fixed and stained by the TUNEL method.

FIG. 7 demonstrates, in accordance with an embodiment of the invention, the effect of RA on activation of HSCs and MFs in BDL-induced liver injury. A. Isolation of pure HSCs from BDL-induced fibrotic livers by FACS. The Coll-GFP mice were subjected to sham or BDL operation. NPCs isolated from these mice were subjected to FACS using 350 nm (vitamin A) and 488 nm (GFP) lasers. BSCs lacking vitamin A lipids and GFP expression were used as negative control. Boxed areas show the percentage of the GFP− or GFP+HSCs in the UV+/vitamin A+ fraction. For qPCR analysis, all UV+/vitamin A+ HSCs were sorted. B. qPCR analysis of vitamin A+ HSCs isolated from sham or BDL mice reveals significant increases in mRNA expression of Colla1, Sma, and Timp1 in vitamin A+HSCs. **P<0.01. C and D. Effects of RA treatment on expression of SMA in PFs around the bile ducts and HSCs in the sinusoid in BDL mice. Immunohistochemistry of Desmin and SMA were analyzed for both MFs around the portal vein and HSCs in the sinusoid. Arrows indicate SMA+Desmin+PFs or HSCs. Bar, 10 μm. bd, bile duct; ha, hepatic artery; pv, portal vein. Quantification of the percentage of SMA+Desmin+MFs or HSCs, demonstrates significant attenuation of BDL-induced increased in activation of MFs and HSCs by RA treatment. The density of Desmin+HSCs increases by BDL but RA treatment has not effect on this increase. For this analysis, 3 images were randomly captured in 3 different sections from 3 independent mice in 3 groups and SMA+ and desmin+PFs or HSCs were counted. *P<0.05, **P<0.01. n.s., no significant difference.

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., J. Wiley & Sons (New York, N.Y. 2001); March, Advanced Organic Chemistry Reactions, Mechanisms and Structure 5^{th} ed., J. Wiley & Sons (New York, N.Y. 2001); and Sambrook and Russel, Molecular Cloning: A Laboratory Manual 3rd ed., Cold Spring Harbor Laboratory Press (Cold Spring Harbor, N.Y. 2001), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For purposes of the present invention, the certain terms are defined below.

As used herein, the acronym “HSCs” means hepatic stellate cells.

As used herein, the acronym “PPARγ” means peroxisomal proliferator-activated receptor γ.

As used herein, the acronym “YGW” means Yang-Gan-Wan. As used herein, the acronym “RA” means rosmarinic acid. As used herein, the acronym “BC” means baicalin. As used herein, “beneficial results” may include, but are in no way limited to, lessening or alleviating the severity of the disease condition, preventing the disease condition from worsening, curing the disease condition, preventing the disease condition from developing, lowering the chances of a subject developing the disease condition and prolonging a subject's life or life expectancy.

“Conditions” and “disease conditions,” as used herein may include but are in no way limited to any form of liver fibrosis and cirrhosis.

“Mammal” as used herein refers to any member of the class Mammalia, including, without limitation, humans and nonhuman primates such as chimpanzees and other apes and monkey species; farm animals such as cattle, sheep, pigs, goats and horses; domesticated mammals such as dogs and cats; laboratory animals including rodents such as mice, rats and guinea pigs, and the like. The term does not denote a particular age or sex. Thus, adult and newborn subjects, whether male or female, are intended to be included within the scope of this term.

“Treatment” and “treating,” as used herein refer to both therapeutic treatment and prophylactic or preventative measures, wherein the object is to slow down (lessen) the targeted pathologic condition, prevent the pathologic condition, pursue or obtain beneficial results, or lower the chances of the individual developing the condition even if the treatment is ultimately unsuccessful. Those in need of treatment include those already with the condition as well as those prone to have the condition or those in whom the condition is to be prevented.

Hepatic stellate cells (HSCs) undergo myofibroblastic trans-differentiation (activation) to participate in liver fibrosis, and identification of molecular targets for this cell fate regulation is important for development of efficacious therapeutic modalities for the disease.

The present invention teaches YGW prevents and reverses HSC activation. The inventors' experimentation indicates that YGW functions, at least in part, by epigenetic de-repression of Pparγ involving reductions in MeCP2 expression and its recruitment to the Pparγ promoter, suppressed expression of PRC2 methyltrasferase EZH2 and consequent reduction of H2K27 dimethylation at the 3′ exon. Furthermore, HPLC/MS and NMR analyses identify polyphenolic rosmarinic acid (RA) and baicalin (BC) as active phytocompounds with the therapeutic effects of preventing and reversing HSC activation. RA and BC suppress the expression of and signaling by canonical Wnts, which are implicated in the aforementioned Pparγ epigenetic repression. The inventors also demonstrate herein that RA treatment in mice with existing cholestatic liver fibrosis inhibits HSC activation and progression of liver fibrosis. Overall, the inventors' results demonstrate the therapeutic benefit of YGW, including active components RA and BC for liver fibrosis via Pparγ de-repression, mediated by suppression of canonical Wnt signaling in HSCs.

In certain embodiments, the invention teaches a method for treating and/or inhibiting liver fibrosis in a subject, including providing a therapeutically effective amount of a composition that inhibits epigenetic repression of Pparγ to the subject. In some embodiments, the composition includes Yang-Gan-Wan. In some embodiments, the composition includes Angelica Sinensis, Paeoniae Albiflora, Radix Rehmannae Preparate, and Ligustici Wallichii Rhizoma. In some embodiments, the composition includes rosmarinic acid. In some embodiments, the rosmarinic acid is between 1% and 100% of the total active ingredients. In some embodiments, the rosmarinic acid is between 5% and 50% of the total active ingredients. In some embodiments, the rosmarinic acid is between 10% and 40% of the total active ingredients. In some embodiments, the rosmarinic acid is between 20% and 30% of the total active ingredients. In some embodiments, the composition includes baicalin. In some embodiments, the baicalin is between 1% and 100% of the total active ingredients. In some embodiments, the baicalin is between 5% and 50% of the total active ingredients. In some embodiments, the baicalin is between 10% and 40% of the total active ingredients. In some embodiments, the baicalin is between 20% and 30% of the total active ingredients. In some embodiments, the composition includes biacalin and rosmarinic acid. In certain embodiments, the composition reduces the level of MeCP2 in the subject. In some embodiments, the invention teaches the use of synthetic versions, pharmaceutical equivalents, analogs, derivatives and salts of the individual substances disclosed herein. In some embodiments, the subject is a mammal. In some embodiments, the subject is a human.

In certain embodiments, the invention teaches a method for reducing or eliminating the symptoms of liver fibrosis in a subject, including providing a therapeutically effective amount of a composition that inhibits epigenetic repression of Pparγ to the subject. In some embodiments, the composition includes Yang-Gan-Wan. In some embodiments, the composition includes Angelica Sinensis, Paeoniae Albiflora, Radix Rehmannae Preparate, and Ligustici Wallichii Rhizoma. In some embodiments, the composition includes rosmarinic acid. In some embodiments, the rosmarinic acid is between 1% and 100% of the total active ingredients. In some embodiments, the rosmarinic acid is between 5% and 50% of the total active ingredients. In some embodiments, the rosmarinic acid is between 10% and 40% of the total active ingredients. In some embodiments, the rosmarinic acid is between 20% and 30% of the total active ingredients. In some embodiments, the composition includes baicalin. In some embodiments, the baicalin is between 1% and 100% of the total active ingredients. In some embodiments, the baicalin is between 5% and 50% of the total active ingredients. In some embodiments, the baicalin is between 10% and 40% of the total active ingredients. In some embodiments, the baicalin is between 20% and 30% of the total active ingredients. In some embodiments, the composition includes biacalin and rosmarinic acid. In certain embodiments, the composition reduces the level of MeCP2 in the subject. In some embodiments, the invention teaches the use of synthetic versions, pharmaceutical equivalents, analogs, derivatives and salts of the individual substances disclosed herein. In some embodiments, the subject is a mammal. In some embodiments, the subject is a human.

In certain embodiments, the invention teaches a method for reducing the likelihood that a subject will develop liver fibrosis, including administering a therapeutically effective amount of a composition that inhibits epigenetic repression of Pparγ to the subject. In some embodiments, the composition includes Yang-Gan-Wan. In some embodiments, the composition includes Angelica Sinensis, Paeoniae Albiflora, Radix Rehmannae Preparate, and Ligustici Wallichii Rhizoma. In some embodiments, the composition includes rosmarinic acid. In some embodiments, the rosmarinic acid is between 1% and 100% of the total active ingredients. In some embodiments, the rosmarinic acid is between 5% and 50% of the total active ingredients. In some embodiments, the rosmarinic acid is between 10% and 40% of the total active ingredients. In some embodiments, the rosmarinic acid is between 20% and 30% of the total active ingredients. In some embodiments, the composition includes baicalin. In some embodiments, the baicalin is between 1% and 100% of the total active ingredients. In some embodiments, the baicalin is between 5% and 50% of the total active ingredients. In some embodiments, the baicalin is between 10% and 40% of the total active ingredients. In some embodiments, the baicalin is between 20% and 30% of the total active ingredients. In some embodiments, the composition includes biacalin and rosmarinic acid. In certain embodiments, the composition reduces the level of MeCP2 in the subject. In some embodiments, the invention teaches the use of synthetic versions, pharmaceutical equivalents, analogs, derivatives and salts of the substances disclosed herein. In some embodiments, the subject is a mammal. In some embodiments, the subject is a human.

In certain embodiments, the invention teaches a method for screening for compositions that, when administered to a subject, are effective in one or more of: treating, inhibiting, promoting the prophylaxis of, preventing, alleviating the symptoms of, and reducing the likelihood of liver fibrosis. In certain embodiments, the screening is accomplished by testing for compositions and/or compounds that are effective in inhibiting epigenetic repression of Pparγ and/or reducing the level of MeCP2 in the subject. In some embodiments, the subject is a mammal. In some embodiments, the subject is a human.

In various embodiments, the invention teaches a composition that inhibits epigenetic repression of Pparγ when administered to a subject. In some embodiments, the composition includes Yang-Gan-Wan. In some embodiments, the composition includes Angelica Sinensis, Paeoniae Albiflora, Radix Rehmannae Preparate, and Ligustici Wallichii Rhizoma. In some embodiments, the composition includes rosmarinic acid. In some embodiments, the rosmarinic acid is between 1% and 100% of the total active ingredients. In some embodiments, the rosmarinic acid is between 5% and 50% of the total active ingredients. In some embodiments, the rosmarinic acid is between 10% and 40% of the total active ingredients. In some embodiments, the rosmarinic acid is between 20% and 30% of the total active ingredients. In some embodiments, the composition includes baicalin. In some embodiments, the baicalin is between 1% and 100% of the total active ingredients. In some embodiments, the baicalin is between 5% and 50% of the total active ingredients. In some embodiments, the baicalin is between 10% and 40% of the total active ingredients. In some embodiments, the baicalin is between 20% and 30% of the total active ingredients. In some embodiments, the composition includes biacalin and rosmarinic acid.

In various embodiments, one or more compositions or compounds disclosed herein may be provided as a pharmaceutical composition, including a pharmaceutically acceptable excipient along with a therapeutically effective amount of one or more of the compounds or compositions described herein. “Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients may be solid, liquid, semisolid, or, in the case of an aerosol composition, gaseous.

In various embodiments, the pharmaceutical compositions according to the invention may be formulated for delivery via any route of administration. “Route of administration” may refer to any administration pathway known in the art, including but not limited to aerosol, nasal, oral, transmucosal, transdermal or parenteral. “Transdermal” administration may be accomplished using a topical cream or ointment or by means of a transdermal patch. “Parenteral” refers to a route of administration that is generally associated with injection, including intraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Via the parenteral route, the compositions may be in the form of solutions or suspensions for infusion or for injection, or as lyophilized powders. Via the enteral route, the pharmaceutical compositions can be in the form of tablets, gel capsules, sugar-coated tablets, syrups, suspensions, solutions, powders, granules, emulsions, microspheres or nanospheres or lipid vesicles or polymer vesicles allowing controlled release. Via the topical route, the pharmaceutical compositions based on compounds according to the invention may be formulated for treating the skin and mucous membranes and are in the form of ointments, creams, milks, salves, powders, impregnated pads, solutions, gels, sprays, lotions or suspensions. They can also be in the form of microspheres or nanospheres or lipid vesicles or polymer vesicles or polymer patches and hydrogels allowing controlled release. These topical-route compositions can be either in anhydrous form or in aqueous form depending on the clinical indication.

The pharmaceutical compositions according to the invention can also contain any pharmaceutically acceptable carrier. “Pharmaceutically acceptable carrier” as used herein refers to a pharmaceutically acceptable material, composition, or vehicle that is involved in carrying or transporting one or more compositions or molecules of interest from one tissue, organ, or portion of the body to another tissue, organ, or portion of the body. For example, the carrier may be a liquid or solid filler, diluent, excipient, solvent, or encapsulating material, or a combination thereof. Each component of the carrier must be “pharmaceutically acceptable” in that it must be compatible with the other ingredients of the formulation. It must also be suitable for use in contact with any tissues or organs with which it may come in contact, meaning that it must not carry a risk of toxicity, irritation, allergic response, immunogenicity, or any other complication that excessively outweighs its therapeutic benefits.

The pharmaceutical compositions according to the invention can also be encapsulated, tableted or prepared in an emulsion or syrup for oral administration. Pharmaceutically acceptable solid or liquid carriers may be added to enhance or stabilize the composition, or to facilitate preparation of the composition. Liquid carriers include syrup, peanut oil, olive oil, glycerin, saline, alcohols and water. Solid carriers include starch, lactose, calcium sulfate, dihydrate, terra alba, magnesium stearate or stearic acid, talc, pectin, acacia, agar or gelatin. The carrier may also include a sustained release material such as glyceryl monostearate or glyceryl distearate, alone or with a wax.

The pharmaceutical preparations are made following the conventional techniques of pharmacy involving milling, mixing, granulation, and compressing, when necessary, for tablet forms; or milling, mixing and filling for hard gelatin capsule forms. When a liquid carrier is used, the preparation will be in the form of syrup, elixir, emulsion or an aqueous or non-aqueous suspension. Such a liquid formulation may be administered directly p.o. or filled into a soft gelatin capsule.

The pharmaceutical compositions and molecules according to the invention may be delivered in a therapeutically effective amount. The precise therapeutically effective amount is that amount of the composition or molecule that will yield the most effective results in terms of efficacy of treatment in a given subject. This amount will vary depending upon a variety of factors, including but not limited to the characteristics of the therapeutic composition or molecule (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, for instance, by monitoring a subject's response to administration of a composition or molecule and adjusting the dosage accordingly. For additional guidance, see Remington: The Science and Practice of Pharmacy (Gennaro ed. 20th edition, Williams & Wilkins PA, USA) (2000).

Typical dosages of an effective amount of any of the compositions or molecules described herein can be as indicated to the skilled artisan by the in vitro responses or responses in animal models. Such dosages typically can be reduced by up to about one order of magnitude in concentration or amount without losing the relevant biological activity. Thus, the actual dosage will depend upon the judgment of the physician, the condition of the patient, and the effectiveness of the therapeutic method.

In some embodiments, a therapeutic dosage range of YGW is between 1 mg/kg and 1000 mg/kg of body weight per day when taken orally. In some embodiments, a therapeutic dosage range of YGW is between 5 mg/kg and 700 mg/kg of body weight per day when taken orally. In some embodiments, a therapeutic dosage range of YGW is between 10 mg/kg and 600 mg/kg of body weight per day when taken orally. In some embodiments, a therapeutic dosage range of YGW is between 50 mg/kg and 400 mg/kg of body weight per day when taken orally. In some embodiments, a therapeutic dosage range of YGW is between 100 mg/kg and 200 mg/kg of body weight per day when taken orally. In an embodiment, a therapeutic dosage of YGW is 5 mg/kg of body weight per day when taken orally.

In some embodiments, a therapeutic dosage range of YGW is between 1 mg/kg and 1000mg/kg of body weight per day when taken intravenously. In some embodiments, a therapeutic dosage range of YGW is between 5 mg/kg and 700 mg/kg of body weight per day when taken intravenously. In some embodiments, a therapeutic dosage range of YGW is between 10 mg/kg and 600 mg/kg of body weight per day when taken intravenously. In some embodiments, a therapeutic dosage range of YGW is between 50 mg/kg and 400 mg/kg of body weight per day when taken intravenously. In some embodiments, a therapeutic dosage range of YGW is between 100 mg/kg and 200 mg/kg of body weight per day when taken intravenously. In an embodiment, a therapeutic dosage of YGW is 5 mg/kg of body weight per day when taken intravenously.

In some embodiments, a therapeutic dosage range of RA is between 1 mg/kg and 1000 mg/kg of body weight per day when taken orally. In some embodiments, a therapeutic dosage range of RA is between 5 mg/kg and 700 mg/kg of body weight per day when taken orally. In some embodiments, a therapeutic dosage range of RA is between 10 mg/kg and 600 mg/kg of body weight per day when taken orally. In some embodiments, a therapeutic dosage range of RA is between 50 mg/kg and 400 mg/kg of body weight per day when taken orally. In some embodiments, a therapeutic dosage range of RA is between 100 mg/kg and 200 mg/kg of body weight per day when taken orally. In an embodiment, a therapeutic dosage of RA is 5 mg/kg of body weight per day when taken orally.

In some embodiments, a therapeutic dosage range of RA is between 1 mg/kg and 1000 mg/kg of body weight per day when taken intravenously. In some embodiments, a therapeutic dosage range of RA is between 5 mg/kg and 700 mg/kg of body weight per day when taken intravenously. In some embodiments, a therapeutic dosage range of RA is between 10 mg/kg and 600 mg/kg of body weight per day when taken intravenously. In some embodiments, a therapeutic dosage range of RA is between 50 mg/kg and 400 mg/kg of body weight per day when taken intravenously. In some embodiments, a therapeutic dosage range of RA is between 100 mg/kg and 200 mg/kg of body weight per day when taken intravenously. In an embodiment, a therapeutic dosage of RA is 5 mg/kg of body weight per day when taken intravenously.

In some embodiments, a therapeutic dosage range of BC is between 1 mg/kg and 1000 mg/kg of body weight per day when taken orally. In some embodiments, a therapeutic dosage range of BC is between 5 mg/kg and 700 mg/kg of body weight per day when taken orally. In some embodiments, a therapeutic dosage range of BC is between 10 mg/kg and 600 mg/kg of body weight per day when taken orally. In some embodiments, a therapeutic dosage range of BC is between 50 mg/kg and 400 mg/kg of body weight per day when taken orally. In some embodiments, a therapeutic dosage range of BC is between 100 mg/kg and 200 mg/kg of body weight per day when taken orally. In an embodiment, a therapeutic dosage of BC is 5 mg/kg of body weight per day when taken orally.

In some embodiments, a therapeutic dosage range of BC is between 1 mg/kg and 1000 mg/kg of body weight per day when taken intravenously. In some embodiments, a therapeutic dosage range of BC is between 5 mg/kg and 700 mg/kg of body weight per day when taken intravenously. In some embodiments, a therapeutic dosage range of BC is between 10 mg/kg and 600 mg/kg of body weight per day when taken intravenously. In some embodiments, a therapeutic dosage range of BC is between 50 mg/kg and 400 mg/kg of body weight per day when taken intravenously. In some embodiments, a therapeutic dosage range of BC is between 100 mg/kg and 200 mg/kg of body weight per day when taken intravenously. In an embodiment, a therapeutic dosage of BC is 5 mg/kg of body weight per day when taken intravenously.

The present invention also teaches a kit directed to one or more of: treating, inhibiting, promoting the prophylaxis of and/or preventing, alleviating the symptoms of, and reducing the likelihood of liver fibrosis, in a mammal in need thereof. The kit is an assemblage of materials or components, including at least one of the inventive compositions or molecules described herein. Thus, in some embodiments the kit contains a composition including one or more of: YGW, RA, BC and combinations thereof. The kit may alternatively contain one or more analog, derivative, salt, synthetic version or pharmaceutical equivalent of any of the substances described herein.

The exact nature of the components configured in the inventive kit depends on its intended purpose. By way of non-limiting example, some embodiments are configured for one or more purpose selected from: treating, inhibiting, promoting the prophylaxis of and/or preventing, alleviating the symptoms of, and/or reducing the likelihood of liver fibrosis. In one embodiment, the kit is configured particularly for the purpose of treating mammalian subjects. In another embodiment, the kit is configured particularly for the purpose of treating human subjects. In another embodiment, the kit is configured for treating adolescent, child, or infant human subjects. In further embodiments, the kit is configured for veterinary applications, treating subjects such as, but not limited to, farm animals, domestic animals, and laboratory animals.

Instructions for use may be included in the kit. “Instructions for use” typically include a tangible expression describing the technique to be employed in using the components of the kit to effect a desired outcome, such as treating, inhibiting, promoting the prophylaxis of and/or preventing, alleviating the symptoms of, and/or reducing the likelihood of liver fibrosis. Optionally, the kit also contains other useful components, such as, diluents, buffers, pharmaceutically acceptable carriers, syringes, catheters, applicators, pipetting or measuring tools, bandaging materials or other useful paraphernalia as will be readily recognized by those of skill in the art.

The materials or components assembled in the kit can be provided to the practitioner stored in any convenient and suitable ways that preserve their operability and utility. For example the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions, molecules and the like. The packaging material is constructed by well-known methods, preferably to provide a sterile, contaminant-free environment. As used herein, the term “package” refers to a suitable solid matrix or material such as glass, plastic, paper, foil, and the like, capable of holding the individual kit components. Thus, for example, a package can be one or more glass vials or plastic containers used to contain suitable quantities of an inventive composition disclosed herein. The packaging material generally has an external label which indicates the contents and/or purpose of the kit and/or its components.

One skilled in the art will recognize many methods and materials similar or equivalent to those described herein, which could be used in the practice of the present invention. Indeed, the present invention is in no way limited to the methods and materials described.

### EXAMPLES

### Example 1

### Introduction

Excessive scarring of the liver results in cirrhosis, the end-stage liver disease of high mortality for which efficacious medical treatments are not currently available, except for liver transplantation. Central to the pathogenesis of the disease is trans-differentiation or activation of hepatic stellate cells (HSCs), vitamin-A storing liver pericytes, into myofibroblastic cells with increased capacity for extracellular matrix (ECM) production and contractility. For better understanding of HSC trans-differentiation, primary efforts have been made on gene regulation and intracellular signaling for expression of activation-associated molecules such as collagens, cytokines (TGF-β, PDGF), chemokines (MCP-1), ECM degradation enzymes and inhibitors (MMPs, TIMPs), NADPH oxidase, renin-angiotensin system, and TLR4. Yet, fundamental questions concerning cell fate regulation of HSCs, remain largely underexplored.

HSCs express many neuronal or glial cell markers, and their neuroectoderm origin was proposed with a subsequent failure to validate this notion using the Wnt1-Cre and ROSA26 reporter mice. This finding logically favored a hypothesis of mesoderm-derived multipotent mesenchymal progenitor cells (MMPC) as the origin of HSCs since MMPC also give rise to neural cells besides other mesenchymal lineages for smooth muscle cells, chondrocytes, osteoblasts, and adipocytes whose markers are also expressed by HSCs. Inconsistent with this notion, at least one recent study demonstrates the mesoderm origin of mouse fetal HSCs.

A fat-storing phenotype is a unique and distinct feature of quiescent HSCs, and it was proposed a decade ago that there is a regulatory commonality between adipocytes and quiescent HSCs. Germane to this proposal is the expression and regulation by the master adipogenic transcription factor PPARγ, which is essential for both adipocyte differentiation and HSC quiescence. PPARγ promotes storage of intracellular fat including retinyl esters in HSCs while suppressing α1(I) collagen promoter via inhibition of p300-facilitated NF-I binding. As shown for inhibition of adipogenesis, canonical Wnt signaling suppresses the expression and promoter activation of Pparγ in HSC trans-differentiation. Necdin, a member of the melanoma antigen family (MAGE) of proteins, inhibits differentiation of adipocytes but promotes that of neurons, skeletal and smooth muscle cells. A recent study demonstrates Wnt10b, one of canonical Wnts expressed by activated HSCs, is a direct target of necdin and the necdin-Wnt pathway causes HSC trans-differentiation via epigenetic repression of Pparγ. This epigenetic regulation involves induction and recruitment of the methyl-CpG binding protein MeCP2 to the Pparγ promoter and concomitant H3K27 di- and tri-methylation in the 3′ exons of Pparγ, resulting in formation of a repressive chromatin structure. It has been demonstrated MeCP2-mediated induction of EZH2, a H3K27 methyltransferase of the polycomb repressive complex 2 (PRC2), is responsible for H3K27 di- and tri-methylation. Most recently, this paradigm of the MeCP2-EZH2 regulatory relay has been characterized in neuronal differentiation where MeCP2-mediated epigenetic repression of miR137 is shown to result in EZH2 induction.

This epigenetic mechanism of Pparγ repression involving the MeCP2-EZH2 relay identifies potential new therapeutic targets for liver fibrosis. To this end, the inventors demonstrate herein that the herbal mixture Yang-Gan-Wan (YGW) targets and abrogates the MeCP2-EZH2 relay of epigenetic Pparγ repression to reverse activated HSCs to their quiescent phenotype. The inventors' HPLC-MS and NMR analyses, coupled with bioassays with primary HSCs, identified rosmarinic acid (RA) and baicalin (BC) as active phytocompounds of YGW.

### Example 2

### Animal Experiments

Male C57B1/6 and collagen α1(I) promoter-GFP (Coll-GFP) mice were subjected to ligation and scission of the common bile duct (BDL) to induce cholestatic liver fibrosis for HSC isolation or testing the therapeutic efficacy of RA. For the latter, after one week following BDL, RA was intraperitoneally injected daily at the dose of 0.1 mg/25 g body weight until the animals were sacrificed one week later for Sirius-red staining morphometry, immunohistochemistry, and qPCR analysis of the livers as described below.

### Example 3

### Hepatic Stellate Cell Isolation and Culture

HSCs were isolated from normal male Wistar rats, C57B1/6 and Coll-GFP mice by in situ digestion of the liver and arabinogalactan gradient ultracentrifugation by the Non-Parenchymal Liver Cell Core of the Southern California Research Center for ALPD and Cirrhosis as described previously in Cheng J H et al,. Wnt antagonism inhibits hepatic stellate cell activation and liver fibrosis, Am J Physiol Gastrointest Liver Physiol, 2007 Nov 15; and Zhu N L et al., The Necdin-Wnt pathway causes epigenetic peroxisome proliferator-activated receptor gamma repression in hepatic stellate cells, J Biol Chem, 2010 Oct 1; 285(40):30463-30471, both of which are incorporated by reference herein in their entirety as though fully set forth. The purity of the cells, as determined by phase contrast microscopy and ultraviolet-excited fluorescence microscopy, exceeded 96%, and the viability as determined by trypan blue exclusion exceeded 94%. In vitro activation of HSCs was achieved by culturing rat HSCs in Dulbecco's modified Eagle's medium (DMEM) with 1.0 g/liter glucose, 10% fetal bovine serum and 1% antibiotics on plastic dish for 3, 5 or 7 days. Culture-activated rat primary HSCs were treated with the YGW or starch (control) aqueous extract at 25% (v/v). To obtain the extract, the YGW or starch powder (provided by S.P. Pharmaceutics Inc.) was suspended in DMEM at the concentration of 35 mg/ml, mixed thoroughly with a vortex for 5 min, and centrifuged at ×150 g for 30 min to collect the supernatant. This supernatant was designated as 100% extract and used after filter-sterilization. RA and BC (Sigma Chemical Co) were dissolved in DMSO and tested at the concentration of 67.5˜270 μM.

### Example 4

### Fluorescence Activated Cell Sorting (FACS)

Two weeks after BDL or sham operation, nonparenchymal cells (NPCs) were isolated from the Coll-GFP mice and subjected to FACS using FACS AriaII sorter (BD Bioscience) at the USC-CSCRMNCCC Flow Cytometry Core. GFP expression was analyzed by an argon laser at 488 nm and a 530 nm filter. Vitamin A autofluorescence was analyzed by a solid-state laser at 350 nm and a 450 nm filter. As a negative control for vitamin A autofluorescence, the inventors used the spontaneously immortalized rat HSC line (BSC) established from cholestatic liver fibrosis in rats, as described in Sung C K et al, Tumor necrosis factor-alpha inhibits peroxisome proliferator-activated receptor gamma activity at a posttranslational level in hepatic stellate cells, Am J Physiol Gastrointest Liver Physiol, 2004 May; 286(5):G722-G729, which is incorporated herein by reference as though fully set forth.

### Example 5

### Immunohistochemistry, TUNEL and Lipid Staining

After 3 days of the extract treatment, the cells were washed with cold phosphate-buffered saline (PBS) and fixed in 4% paraformaldehyde (PF). To stain α-smooth muscle actin (SMA), a fluorescein isothiocyanate (FITC) conjugated antibody (1:200, Sigma, Saint Louis, Mo.) was added as a primary antibody at 4° C. for overnight. After washing and blocking with 5% nonfat milk, fluorescence images were viewed by a Nikon microscope as described above. For intracellular lipid staining, HSCs treated with the extract for 3 days were cultured with retinol (5 μM) and palmitic acid (100 μM) (Sigma, Saint Louis, Mo.) for 48 hr, and fixed with 10% formalin in PBS. Oil Red O (0.5% w/v in isopropanol) was diluted with 67% volume of water, filtered, and added to the fixed HSCs. Apoptosis was detected in cultured HSCs and liver sections from BDL mice using a Cell Death Detection kit from Roche. For liver section immunostaining, liver tissues were fixed with 4% PF and embedded in freezing medium. Cryosections (7 μm) were washed with PBS, digested with 20 μg/ml proteinase K (Invitrogen, Carlsbad, Calif.), and blocked with 5% goat serum and 0.2% bovine serum albumin. The sections were then incubated with mouse anti-SMA antibody conjugated with FITC (Sigma, 1:400) and rabbit anti-desmin antibody (Thermo Scientific, Rockford, Ill., 1:400). After washing, the sections were incubated with goat anti-rabbit antibody conjugated with AlexaFluor 568 (Invitrogen, 1:400) and mouse anti-FITC antibody conjugated with DyLight 488 (Jackson ImmunoResearch, West Grove, Pa., 1:400). The sections were mounted with 4,6-diamidino-2-phenylindole (DAPI) (Invitrogen) and fluorescence images were visualized under a microscope. To quantify the percentage and density of HSCs in the liver after BDL with or without treatment of RA, 6 images were randomly captured using a 10× objective lens in 3 different sections and SMA+ and desmin+HSCs in the parenchyma were counted.

### Example 6

### Real Time Quantitative PCR

Total RNA was extracted from the cells using TRIzol reagent (Invitrogen) or RNeasy Mini kit (Qiagen). One microgram of RNA was reverse transcribed to cDNA by using SuperScript III First-Strand Synthesis System (Invitrogen) and amplified by 40 cycles using primers listed below and the SYBR Green PCR Master mix reagent (AB Applied Biosystem). Each threshold cycle (Ct) value was first normalized to the 36B4 Ct value of a sample and subsequently compared between the treatment and control samples. Primer sequences used were:

Pparγ,
SEQ ID NO: 1
5′-CCT GAA GCT CCA AGAATA CCA AA-3′;
and
SEQ ID NO: 2
5′-AGA GTT GGG TTT TTT CAG AAT AAT AAGG;
α1(I)Coll,
SEQ ID NO: 3
5′-TCGATT CAC CTA CAG CAC GC
and
SEQ ID NO: 4
5′- GAC TGT CTT GCC CCA AGT TCC;
36B4,
SEQ ID NO: 5
5′- TTCCCA CTG GCT GAA AAG GT
and
SEQ ID NO: 6
5′-CGC AGC CGC AAA TGC;
Ezh2,
SEQ ID NO: 7
5′-AGT GGA GTGGTG CTG AAG
and
SEQ ID NO: 8
5′-GCC GTC CTT TTT CAG TTG;
Tgfβ 1,
SEQ ID NO: 9
5′-AGA AGT CAC CCG CGTGCTA
and
SEQ ID NO: 10
5′-TGT GTG ATG TCT TTG GTT TTG TCA;
Suz12,
SEQ ID NO: 11:
5′-GTG AAG AAG CCGAAA ATG
and
SEQ ID NO: 12
5′-AAT GTT TTC CTT TTG ATG;
Eed,
SEQ ID NO: 13
5′-ATC CTA TAA CAA TGC AGT
and
SEQ ID NO: 14
5′-TTC ATC TCT GTG CCC TTC;
α-Sma,
SEQ ID NO: 15
5′-TGT GCT GGA CTC TGG AGA TG
and
SEQ ID NO: 16
5′-GAT CAC CTG CCC ATC AGG;
Wnt10b,
SEQ ID NO: 17
5′-CGA GAA TGC GGA TCC ACAA
and
SEQ ID NO: 18
5′-CCG CTT CAG GTT TTC CGTTA;
Wnt3a,
SEQ ID NO: 19
5′-CAT CGC CAG TCA CAT GCA CCT
and
SEQ ID NO: 20
5′-CGT CTA TGC CAT GCG AGC TCA;
Desmin,
SEQ ID NO: 21
5′-CAG GAC CTG CTC AAT GTG
and
SEQ ID NO: 22
5′-GTA GCC TCG CTG CTG ACA ACC TC;
Gapdh,
SEQ ID NO: 23
5′-CTG CCC GTA GAC AAA ATG GT
and
SEQ ID NO: 24
5′-GAA TTT GCC GTG AGT GGA GT;
Sma,
SEQ ID NO: 25
5′-CTG AGC GTG GCT ATT CCT TC
and
SEQ ID NO: 26
5′-CCT CTG CAT CCT GTC AGC AA;
Timp1,
SEQ ID NO: 27
5′-CAG TAA GGC CTG TAG CTG TGC
and
SEQ ID NO: 28
5′-CTC GTT GAT TTC GG GGA AC.

### Example 7

### Transfection and Reporter Assay and IKK Assay

TCF promoter-luciferase construct TOPFLASH (a gift from Dr. Randall Moon, Univ. of Washington, Seattle, Wash.) or a KB luciferase construct was used for transient transfection in the rat primary HSCs by electroporation using the Neon™ Transfection System (Invitrogen). The Renilla pRL-TK construct was used for standardization for transfection efficiency. Cell lysates were analyzed by the dual luciferase assay (Promega) on a luminometer. To assess the activity of IKK, IKK was immunoprecipitated by IKKα antibody and protein G-Sepharose, and the assay was performed at 30° C. for 1 hr in buffer containing 20 mM Tris HCl, pH 7.5, 20 mM MgCl₂, 2 mM dithiothreitol, 20 μM ATP, 2 μg GST-IκBα, and [γ-32P]ATP. The reaction was stopped by addition of Laemmi buffer and was resolved by 10% SDS-PAGE followed by a transfer onto a membrane for imaging.

### Example 8

### Immunoblot Analysis

Whole cell extracts were prepared as previously described in Hazra S. et al., Peroxisome proliferator-activated receptor gamma induces a phenotypic switch from activated to quiescent hepatic stellate cells, J Biol Chem, 2004 Mar 19; 279 (12):11392-11401, which is incorporated by reference herein as though fully set forth. Equal amounts of the extract (20 μg) were separated by 8-15% SDS-PAGE and the proteins were transferred to nitrocellulose membranes (Bio-Rad, Hercules, Calif.). MeCP2, type I collagen, and β-actin were detected by incubating with rabbit polyclonal anti-MeCP2 (1:1000) (Abcam), antitype I collagen (1:4000), and anti-β-actin (1:5000) primary antibodies (Santa Cruz Biotechnology) in TBS (100 mM Tris-HCl, 1.5 M NaCl, pH 7.4) with 5% nonfat milk overnightat 4° C. followed by incubation with horseradish peroxidase conjugated goat anti-rabbit secondary antibodies (1:4000) (Sigma) at room temperature for 2 hr. The antigen-antibody complexes' chemiluminescence was detected by using the ECL detection kit (Pierce).

### Example 9

### Chromatin Immunoprecipitation (ChIP)

For assessing Pparγ epigenetic regulation, carrier ChIP was performed using Raji cells as the source of carrier chromatin. For native ChIP, 20 μg of HSC chromatin was mixed with 80 μg of Raji cell chromatin. For cross-link ChIP, Raji cells (1.4×107 cells) were mixed to HSCs (0.2×106 cells) and fixed with 1% formaldehyde on the rotating platform for 5-10 minutes at room temperature followed by addition of glycine to a final concentration of 0.125M. After lysis of the cells with SDS buffer (1% SDS, 10 mM EDTA, 50 mM Tris-HCl pH8.1) with protease inhibitors, the lysates were sonicated and snap frozen in aliquots. For chromatin IP, diluted samples were first pre-cleared using protein G-agarose beads and then incubated with antibody against Ser2P RNApolyII, MeCP2, H3K27me2, H3K4me2 and H3Kacetylated (Abcam) at 1 μg/μl at 4° C. for overnight followed by precipitation with protein G agarose beads. After elution of immunoprecipitated complex, crosslinking was reversed with 5N NaCl and proteins digested with protease K. Extracted chromatin was subjected to real-time PCR using the primers flanking a segment within Pparγ promoter or exon as previously described in Mann J et al., MeCP2 controls an epigenetic pathway that promotes myofibroblast transdifferentiation and fibrosis, Gastroenterology, 2010 Feb; 138(2):705-14, 714, which is incorporated herein by reference as though fully set forth. Ct values of the samples with non-immune IgG were subtracted and compared to their respective input Ct values.

### Example 10

### Isolation and Identification of Active Compounds

The aqueous YGW extract (350 mg/ml in PBS) was applied to size exclusion chromatography using Super Prep Grade gel in XK 16/70 column (Amersham Pharmacia Biotech, Piscataway, N.J.) and PBS as a mobile phase solvent. The fractions were tested for their bioactivity toward activated HSCs as determined by the morphological reversal of the cells to their quiescence under microscope, and the fractions with the molecular size range of 200-750 Da, were shown to contain the most of the activity. To improve the extraction efficiency of both water soluble and lipophilic phytocompounds and to allow their structural elucidation, n-butanol (BuOH) was added to the YGW water suspension. Briefly, 10 grams of YGW powder suspended in 200 ml of ddH2O were partitioned with 200 ml of BuOH. After centrifugation and phase separation, BuOH and ddH2O were evaporated in vacuo and lyophilized to yield water (3.59 g) and BuOH (0.54 g) soluble part. Based on the bioactivity-guided fractionation, BuOH soluble phytocompounds (500 mg) were fractionated by column chromatography on RP-18 gel (COSMOSIL 75C18-OPN, 20 by 70 mm, Nacalai, USA) eluting with MeCN-H2O mixtures of decreasing polarity. Fraction A (250 ml of 10% MeCNH2O, 192.3 mg), B (250 ml of 40% MeCN-H2O, 196.6 mg), and C (250 ml of 100% MeCN, 64.2 mg) were then subjected to bioassay and high performance liquid chromatographyphotodiode array detection-mass spectrometry (HPLC-DAD-MS) analysis after removing the solvent by using the rotavapor and lyophilizer. HPLC-DAD-MS analysis was carried out on a ThermoFinnigan LCQ Advantage ion trap mass spectrometer with a RP C18 column (Alltech Prevail C18 3 μm 2.1×100 mm) at a flow rate of 125 μl/min with a 10 μl injection. The solvent gradient system and the conditions for MS analysis were as described in Bok J W et al., Chromatin-level regulation of biosynthetic gene clusters, Nat Chem Biol, 2009 Jul; 5(7):462-464, which is incorporated by reference herein in its entirety as though fully set forth. For quantification of RA and BC in each fraction, linear curves of each compound were generated by using extract ion chromatograms (EIC) in negative mode at the molecular weight of each corresponding parent ion. For identification of the major phytocompounds in fraction A, fraction A (135.0 mg) was purified by reverse phase HPLC [Phenomenex Luna 5 μm C18 (2), 250×10 mm] with a flow rate of 5.0 ml/min and measured by a UV detector at 254 nm. The gradient system was MeCN (solvent B) in 5% MeCN/H2O (solvent A) both containing 0.05% TFA: 10% B from 0 to 5 min, 10 to 30% B from 5 to 25 min, 30 to 100% B from 25 to 27 min, 100% B from 27 to 30 min, 100 to 10% B from 30 to 32 min, and re-equilibration with 20% B from 32 to 35 min. RA (4.3 mg) and BC (8.7 mg) were eluted at 22.1 and 23.6 min, respectively. NMR spectral data were collected on a Varian Mercury Plus-400 spectrometer. The structures were elucidated by their mass, 1H-, 13C-, and 2D-NMR data and also confirmed by comparing their spectroscopic data with those described in Lin YL et. al., Nonsteroidal constituents from Solanum incanum L [Abstract], Journal of the Chinese Chemical Society, 2000 Feb 1; (47):247-251; and Tezuka Y, et al., Constituents of roots of Salvia deserta Schang (Xinjiang-Danshen) [Abstract], Chemical and Pharmaceutical Bulletin 1998; (46):107-112, both of which are incorporated herein by reference in their entirety as though fully set forth. Commercial samples from Sigma were also tested for purposes of comparison.

### Example 11

### Data Analysis

Data were presented as the means—S.E. Student's t test was performed to assess the statistical significance between the two sets of data, and p values less than 0.05 were considered significant.

### Example 12

### YGW Reverses Activated HSCs to Quiescent Cells

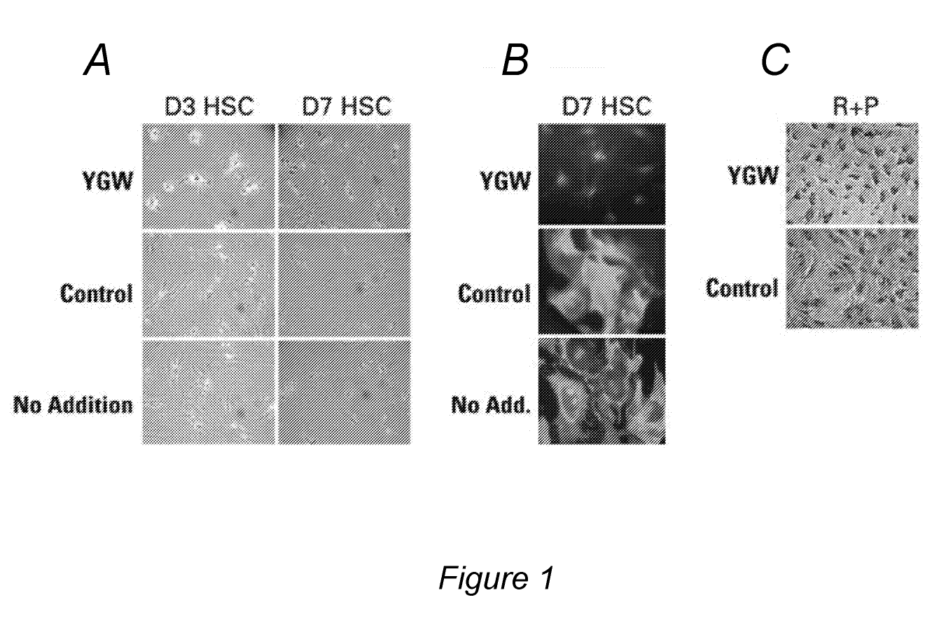

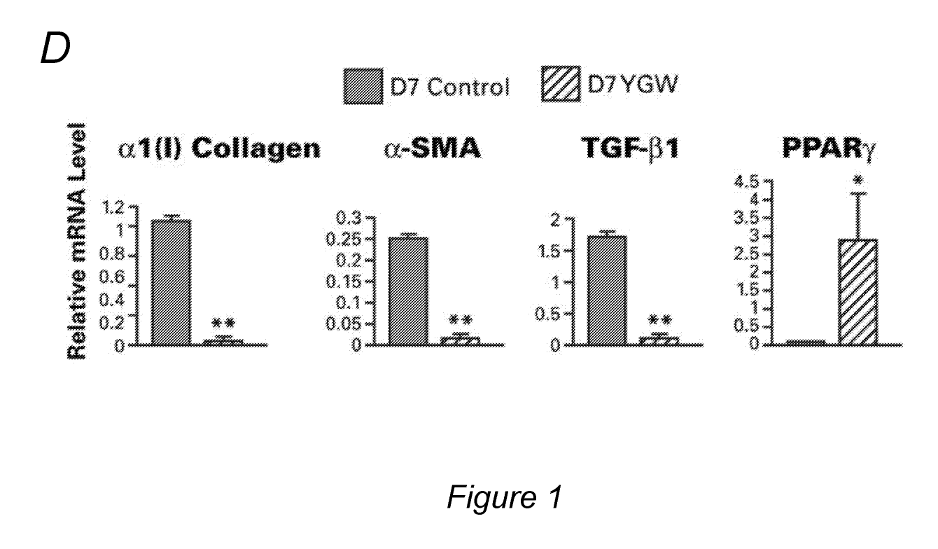

In order to understand the mechanisms of the anti-fibrotic effect of YGW at the cellular level, primary cultures of rat HSCs were treated with the YGW extract or the solvent as a control. Rat HSCs cultured on plastic dish spontaneously undergo myofibroblastic transdifferentiation (“activation”) from day 2˜3 and become fully activated by day 5˜7. Upon treatment of day 3 activating or day 7 fully activated HSCs with the YGW extract for 2 days, activation of HSC is morphologically attenuated as compared to the cells treated with the solvent control or no treatment (FIG. 1A). The YGW decreases the expression of SMA, the bona fide marker for the HSC activation as detected by immunohistochemistry (FIG. 1B) and increases oil red O staining upon addition of retinol and palmitic acid, the parameter for vitamin A storage and the unique feature of quiescent HSCs (FIG. 1C). In addition, the YGW treatment markedly suppresses mRNA expression of markers for HSC activation such as α1(I) procollagen, SMA, and TGF-β1, while upregulating the HSC quiescence marker PPARγ (FIG. 1D). As restored expression of PPARγ reverses activated HSCs to quiescent cells, the observed YGW's effect to prevent or reverse culture-activation of HSCs, is most likely mediated via PPARγ induction.

### Example 13

### YGW Epigenetically De-Represses Pparγ

The epigenetic mechanisms of Pparγ repression in HSC activation involve upregulation and recruitment of the DNA methyl-CpG binding protein MeCP2 to the Pparγ promoter, resulting in the recruitment of the HP-1αco-repressor. MeCP2-dependent upregulation of EZH2, the histone H3 lysine 27 (H3K27) methyltransferase of polychrome repressor complex 2 (PRC2), increases H3K27 di- and trimethylation in the Pparγ exons with consequent formation of a repressive chromatic structure.

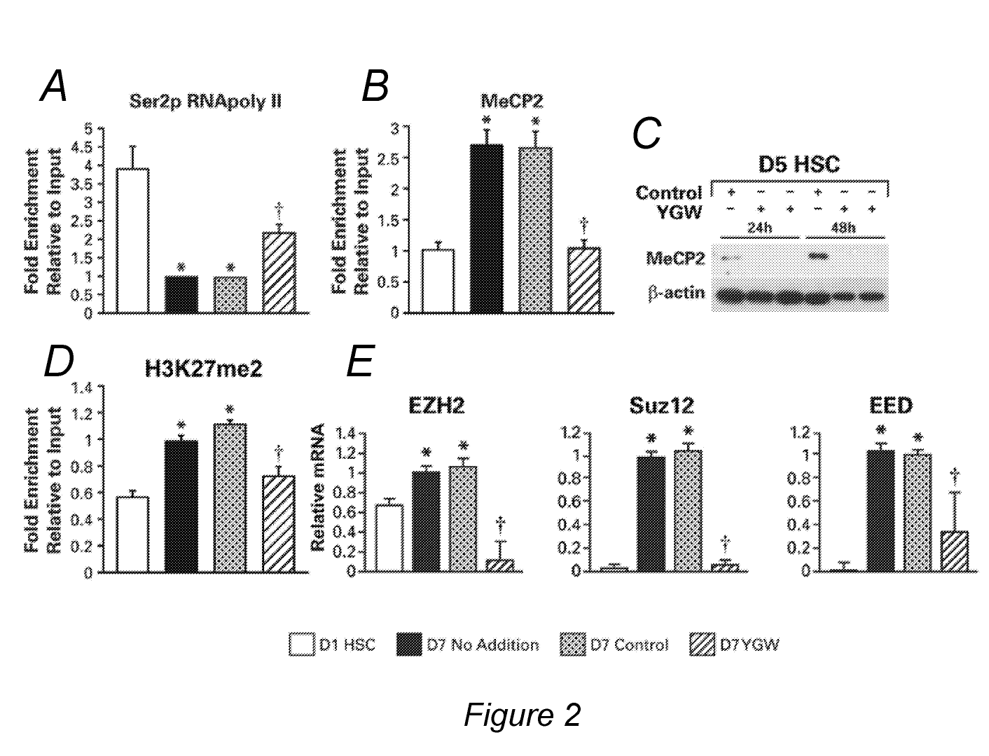

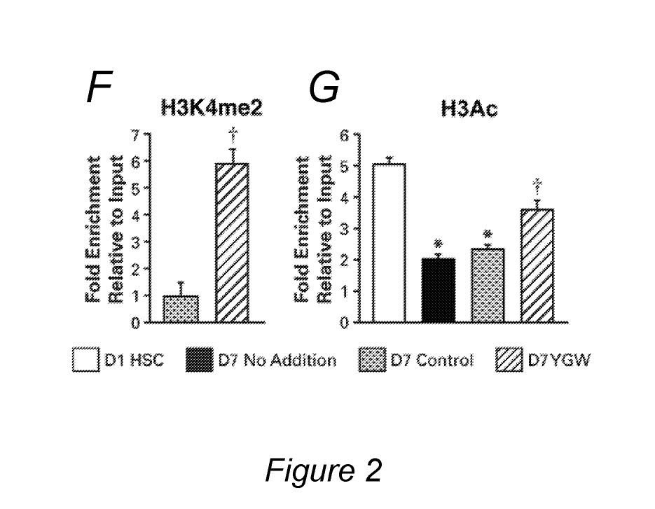

The inventors tested whether YGW's inductive effect on Pparγ is associated with epigenetic effects on this gene. First, the inventors examined the recruitment of elongating RNA polymerase II (Ser2- p RNAPoly II) to the Pparγ gene. Culture-activated HSCs at day 7 have a markedly reduced recruitment of the Ser2-p RNAPoly II as compared to day 1 quiescent HSCs, and this suppression is attenuated by the YGW treatment (FIG. 2A). MeCP2 enrichment to the Pparγ promoter is increased in Day 7 culture-activated HSCs but reduced by the YGW treatment to the level seen in Day 1 HSCs (FIG. 2B). This reduction is associated with abrogation of MeCP2 protein induction seen in day 5 HSCs subsequently incubated with the YGW extract for 24 or 48 hr (FIG. 2C). Increased H3K27 di-methylation (H3K27me2) noted at the exon 2 of Pparγ in culture-activated HSCs, with or without the solvent, is also normalized by the YGW extract (FIG. 2D), most likely attributable to suppressed expression of PRC2 components, EZH2, Suz12, and EED (FIG. 2E). H3K4 di-methylation (H3K4me2) and H3 acetylation (H3Ac), the histone modifications for active transcription, are both increased at the Pparγ promoter locus by the YGW treatment (FIG. 2F and G). These data collectively demonstrate that epigenetic repression of the Pparγ gene in culture-activated HSCs is lifted by the YGW extract treatment, and this effect must be responsible for restored PPARγ expression and HSC quiescence.

### Example 14

YGW Suppresses IKK and NF-κB activity in HSC

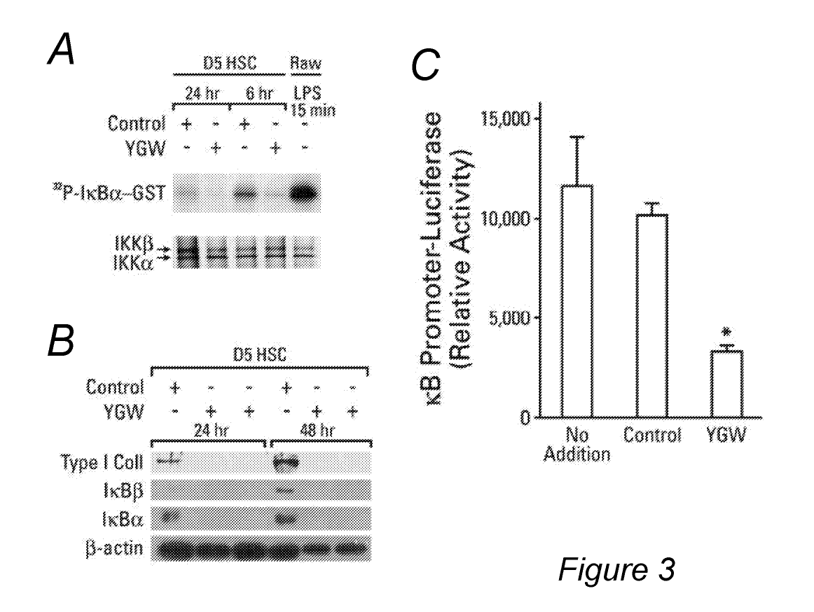

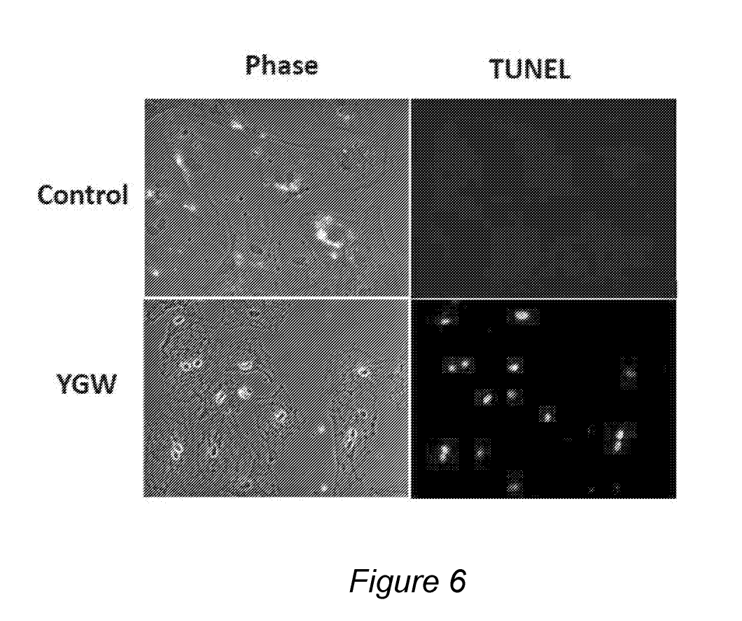

Another important biochemical feature of activated HSCs is increased activity of NF-κB. The inventors tested how the YGW extract affects this parameter. The treatment with the YGW extract markedly inhibits the activity of IκB kinase (IKK) as assessed by phosphorylation of IκBα-GST fusion protein (FIG. 3A), the expression of IκBα and β, both targets of NF-κB (FIG. 3B) in day-5 HSCs, and NF-κB promoter activity in the rat HSC line (BSC) (FIG. 3C). The demonstrated suppressive effects of YGW on IKK and NF-κB suggest that it may promote apoptotic death of HSCs. Only after a prolonged extract treatment exceeding 4-5 days with replenishment of the medium containing the extract every 2 days, apoptosis of cultured HSCs begins to appear and becomes apparent after 8 days as assessed by TUNEL staining (FIG. 6).

### Example 15

### Identification of YGW's Active Ingredients

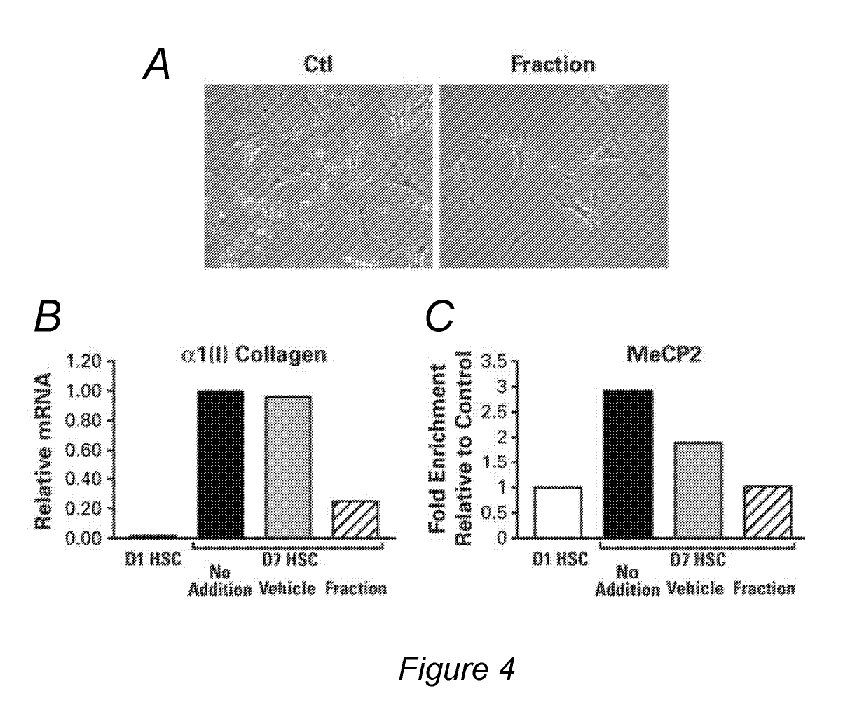

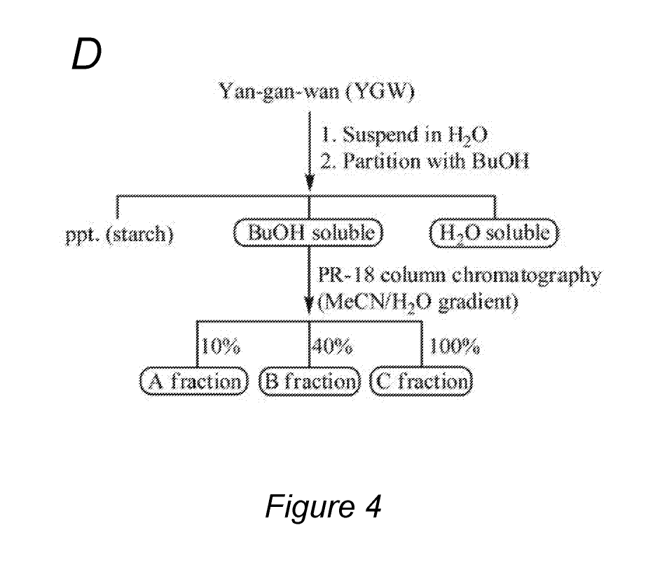

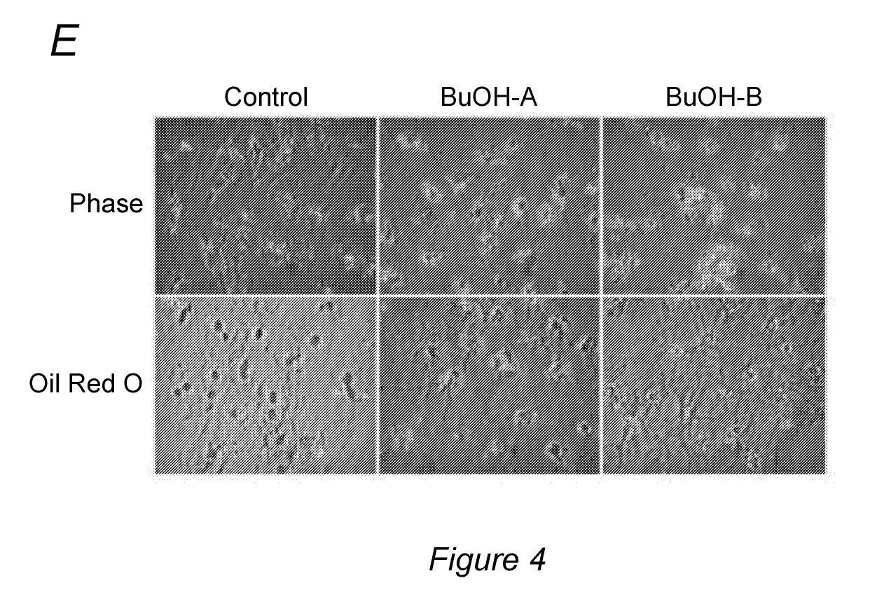

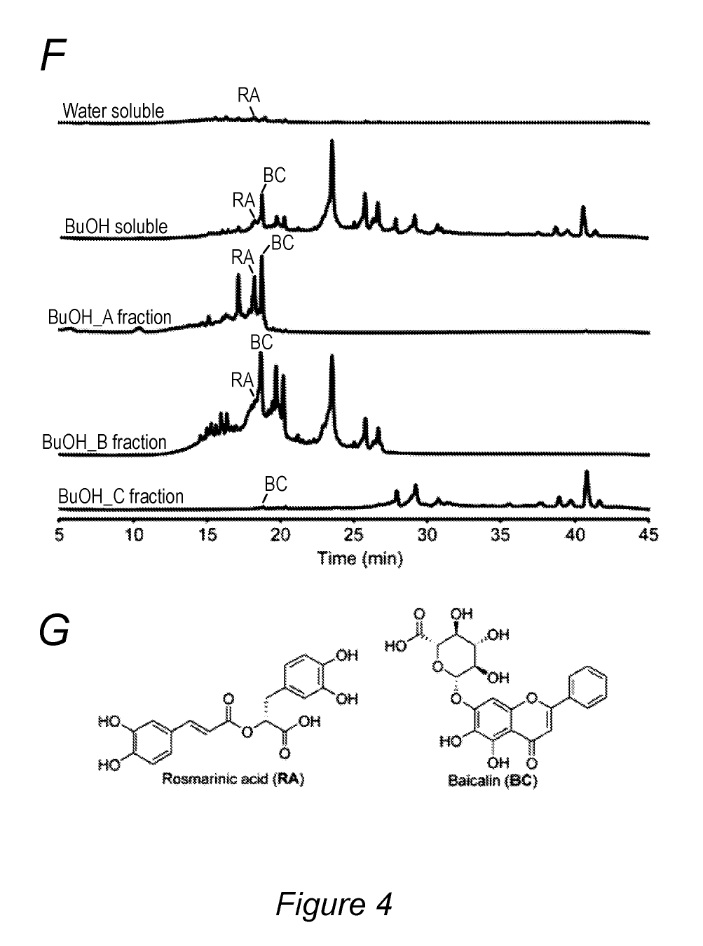

As the first step in identifying active ingredients of YGW rendering the above reversal effects on activated HSCs, the inventors first tested different fractions of gel filtration of the YGW water extract in culture-activated HSCs. This analysis revealed a fraction with a molecular mass range of 200˜750 Da reproduces the YGW effects, including the morphological reversal (FIG. 4A), down regulation of α1(I) procollagen mRNA (FIG. 4B), and decreased MeCP2 enrichment at the Pparγ promoter (FIG. 4C). This gel filtration fraction was next applied to LC/MS for identification of active ingredients. This analysis identified small peaks with the retention time of 14˜15 min (boxed in the UV254 tracing of FIG. 4D). Due to low amounts of these molecules detected in the water extract to allow their purification and identification, the inventors next analyzed YGW ingredients extracted with butanol (BuOH). This method ensures that most hydrophilic and lipophilic organic compounds are extracted into the butanol layer while most of the sugar and ionic inorganic components remain in the water layer. After lyophilization, the water-soluble portion of YGW shows reduced activity of the HSC morphologic reversal when compared with the YGW water extract before the butanol partitioning. In contrast, the butanol-soluble portion of YGW shows clear bioactivity toward HSCs (data not shown), suggesting that the bioactive phytocompounds are enriched in the butanol soluble portion. The inventors further fractionated the butanol soluble portion by reverse phase chromatography eluted with 10% (A fraction), 40% (B fraction), and 100% (C fraction) acetonitrile-water mixtures (FIG. 4D). The butanol A fraction shows a reproducible effect on HSC morphologic reversal (FIG. 4E) while the C fraction causes immediate cytotoxicity evident by detachment of the cells (data not shown). The B fraction shows a moderate reversal effect (FIG. 4E). The HPLC profiles clearly show the metabolites distribution of each fraction and suggest that the bioactive compound(s) may be eluted from 15 min to 20 min in fraction A (FIG. 4F). In order to identify the bioactive phytocompounds in the A fraction, a total of eight subfractions were further purified by semi-preparative HPLC (data not shown). Two major compounds were then isolated and identified to be the bioactive principles. They are rosmarinic acid (RA) and baicalin (BC) (FIG. 4G) by analyzing their mass, 1H-, 13C-, and 2D-NMR data as well as by comparing their 1H-, 13C-NMR data with those of commercial authentic samples (data not shown).

### Example 16

In vitro Effects of RA and BC on HSCs

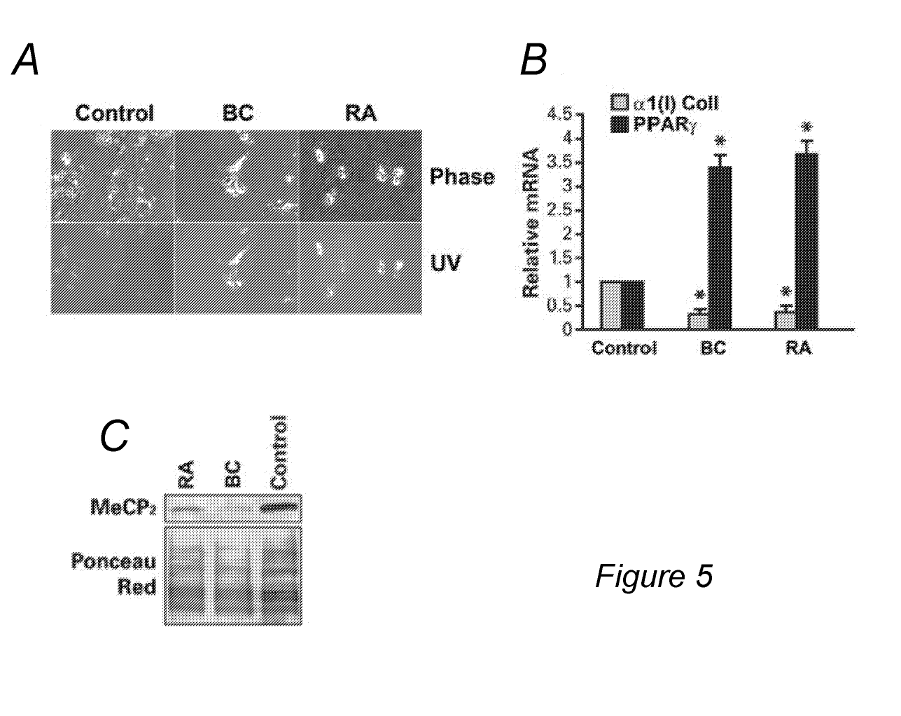

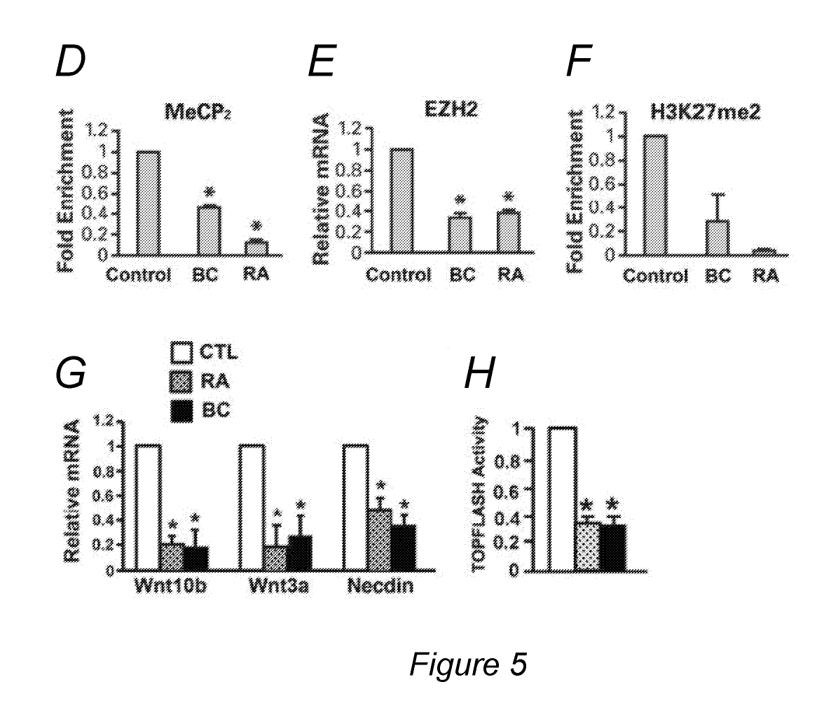

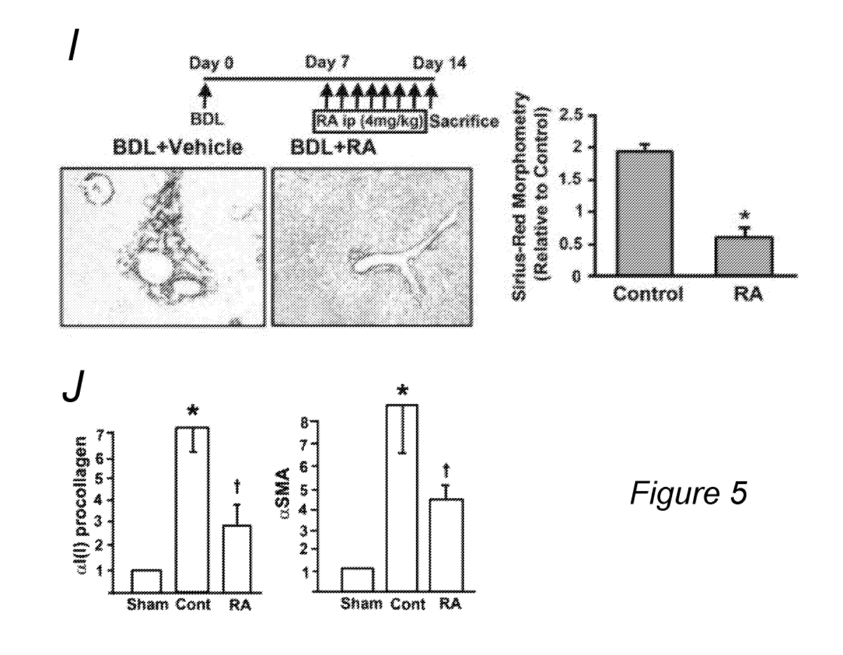

The inventors next tested whether authentic RA and BC reproduce the effects observed with the YGW extract by testing a wide range of concentrations for HSC morphologic reversal. Indeed, both RA and BC morphologically reverse activated HSCs to quiescent cells with increased UV-excited autofluorescence at the concentration of 135 and 270 μM (FIG. 5A). Using the concentration of 270 μM, RA and BC are shown to down-regulate α1(I) procollagen 2˜3 fold and to induce PPARγ 3˜4 fold (FIG. 5B). Both RA and BC reduce MeCP2 protein level (FIG. 5C) and its enrichment in the Pparγ promoter (FIG. 5D). RA and BC also reduce EZH2 expression and H3K27me2 at the Pparγ exon (FIG. 5E and F). Collectively, these results support that RA and BC are indeed active phytocompounds that contribute to YGW's effect to inhibit or reverse HSC activation via epigenetic de-repression of Pparγ.

Both RA and BC suppress the expression of Wnt10b and Wnt3a (FIG. 5G), the canonical Wnts upregulated in HSC activation and TOPFLASH activity (FIG. 5H). Expression of Necdin which transcriptionally upregulates Wnt10b, is also reduced by RA and BC (FIG. 5G), suggesting that these phytocompounds target the Necdin-Wnt-MeCP2 pathway for reversal of HSC activation.

### Example 17

### RA Inhibits HSC Activation and Progression of Biliary Liver Fibrosis in Mice

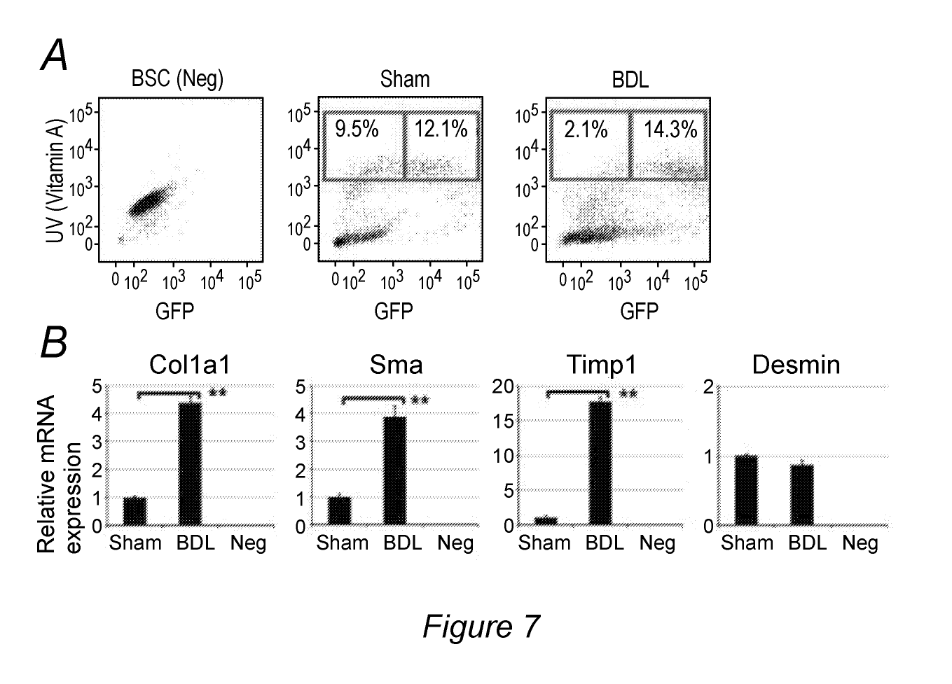

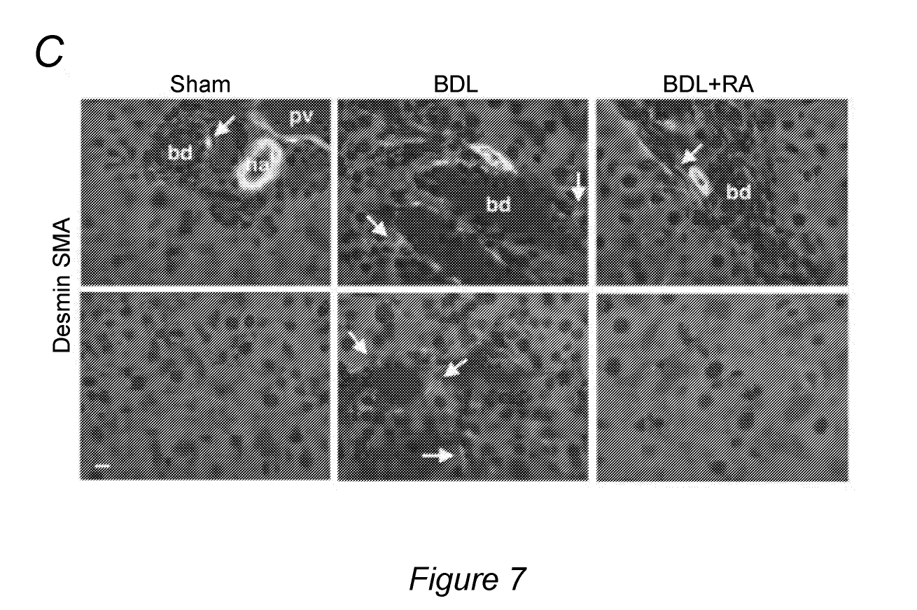

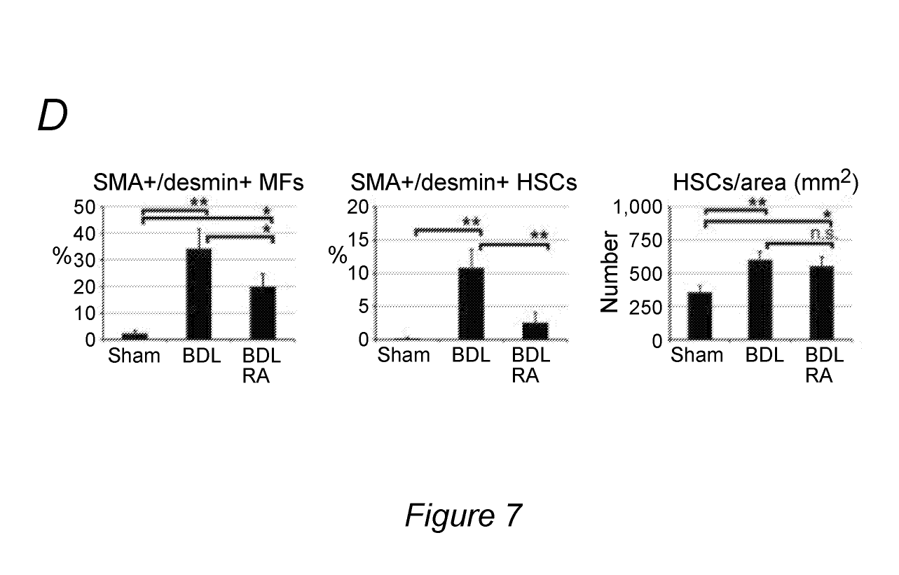

The inventors tested the efficacy of RA for inhibiting progression of pre-existing cholestatic liver fibrosis induced by BDL in mice. As portal myofibroblasts (MFs) rather than HSCs are thought to be the primary source of the fibrotic response in the BDL model, the inventors first examined whether HSCs are activated in the model by analyzing HSCs isolated by FACS from α1l(I) collagen promoter-GFP (Coll-GFP) mice subjected to 2-wk BDL. As shown in FIG. 7A, the percentage of GFP_{low} cells (minimal collagen promoter activity) in UV+(vitamin A containing) HSCs is reduced from 9.5% to 2.1% while the percentage of GFP_{high}/UV+HSCs increases in BDL mice as compared to sham-operated animals, indicating activation of HSCs in the model. Further, qPCR analysis of all UV+HSCs from BDL vs. sham mice, reveals induction of HSC activation markers such as α1(I) procollagen (Co1a1), Sma, and Timp1 in BDL HSCs but not Desmin (FIG. 7). Having confirmed that HSCs are indeed activated in the model, the inventors tested the effects of daily intraperitoneal administration of RA vs. vehicle during the second week of BDL. The liver to body weight percentage is not different between RA or vehicle treated mice (6.8+0.7 vs. 6.3+0.3), nor are the plasma ALT levels (157+71 vs. 283+95, p=0.29). However, the digital morphometric analysis of Sirius red-stained collagen fibers shows a significant attenuation of liver fibrosis by RA treatment (FIG. 5I). To examine whether this antifibrotic effect of RA is associated with suppressed activation of HSCs in vivo, immunohistochemistry for SMA and Desmin were performed (FIG. 7C). In the sham operated liver, expression of SMA is primarily seen in the hepatic artery and a very few cells around the bile duct, but not in HSCs in the sinusoid (FIG. 7C, upper and lower left panel). In the vehicle-treated BDL liver, expression of SMA increases in Desmin+portal MFs and HSCs (FIG. 7C, upper and lower middle panel). RA treatment reduces the percentage of SMA+MFs by 40% and that of SMA+HSCs by 75% (FIG. 7C and 7D). The density of Desmin+HSCs increases by BDL, but RA treatment has no effect on this change (FIG. 7D). No TUNEL+HSCs or hepatocytes are detected in the liver parenchyma of either RA- or vehicle treated BDL livers. These data indicate that RA suppresses activation of both portal MFs and HSCs in BDL-induced liver injury. Hepatic mRNA levels of α1(I) procollagen and SMA are also significantly reduced by RA treatment (FIG. 5J), further supporting anti-fibrotic effects of RA in this model. Taken together, these data indicate that RA suppresses activation of HSCs and liver fibrosis in BDL-induced liver injury.

### Example 18

### Discussion

The present invention demonstrates that the MeCP2 -EZH2 relay of Pparγ epigenetic repression is an important target for the anti-fibrotic effect of the herbal prescription YGW. Polyphenolic RA and flavonoid BC are identified as active phytochemicals in YGW that contribute to the reversal of epigenetic Pparγ repression and the activated phenotype of HSCs. Both RA and BC inhibit MeCP2 induction and its recruitment to the Pparγ promoter while suppressing the expression of PRC2 components including the H3K27 methyltrasferase EZH2 resulting in reduced H3K27me2 at the Pparγ exon locus. These epigenetic effects which result in the formation of euchromatin at the Pparγ locus, increases recruitment of RNA polymerase to Pparγ and its transcription, and restore expression of the gene which is essential for HSC differentiation. In essence, these results provide the molecular basis of the anti-fibrotic effects of YGW and its ingredients RA and BC at the epigenetic level. Due likely to the ability to suppress NF-κB, the prolonged treatment of cultured HSCs with the YGW extract for 8 days causes apoptosis in cultured HSCs (FIG. 6). However, no apoptosis is evident during the first 2 days of the treatment when the epigenetic Pparγ derepression and phenotypic reversal of HSCs are achieved. RA treatment of BDL mice attenuates liver fibrosis, and this effect is accompanied by suppressed activation of HSCs as demonstrated by a marked reduction in SMA+HSCs. In these livers, apoptosis of HSCs is not evident and the number of HSCs is not reduced (FIG. 7C and 7D). Thus, these results suggest that suppressed activation rather than apoptosis of HSCs is responsible at least in part for RA's anti-fibrotic effect in the BDL model. Portal MFs, which are considered as a major source of a fibrogenic response in the BDL model, are indeed increased in number after BDL (FIG. 7D), and this change is attenuated by RA treatment.

Suppression of IKK and NF-κB activities by YGW shown in HSCs is consistent with its ability to suppress oxidant stress, which is a well-known signal for activation of IKK. Oxidant stress generated by NADPH oxidase is recognized as a key signaling event in activation of HSCs, induced by a wide array of agonists such as angiotensin II, PDGF, and leptin. Accordingly, antioxidants which scavenge NADPH oxidase-derived ROS are expected to suppress activation of HSCs. However, the present invention demonstrates that BC and RA inhibit the canonical Wnt signaling which mediates epigenetic repression of Pparγ involving MeCP2 and EZH2. Further, Necdin, which transcriptionally activates Wnt10b via its binding to a GN box in its proximal promoter, is also reduced by both RA and BC. While not wishing to be bound by any one particular theory, taken together these results suggest that both phytocompounds target the Necdin-Wnt-MeCP2-EZH2 pathway for their epigenetic effects.

The various methods and techniques described above provide a number of ways to carry out the application. Of course, it is to be understood that not necessarily all objectives or advantages described can be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some preferred embodiments specifically include one, another, or several features, while others specifically exclude one, another, or several features, while still others mitigate a particular feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

In some embodiments, the terms “a” and “an” and “the” and similar references used in the context of describing a particular embodiment of the application (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. No language in the specification should be construed as indicating any non-claimed element essential to the practice of the application.

Preferred embodiments of this application are described herein, including the best mode known to the inventors for carrying out the application. Variations on those preferred embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

1. A method for treating and/or inhibiting liver fibrosis in a subject, comprising providing a therapeutically effective amount of a composition that inhibits or reduces epigenetic repression of Pparγ to the subject.

2-4. (canceled)

5. The method of claim 1, wherein the composition comprises baicalin.

6. The method of claim1, wherein the composition reduces the level of MeCP2 expression in the subject.

7. The method of claim 1, wherein the composition reduces or eliminates activation of a hepatic stellate cell (HSC) in the subject and/or leads to a quiescent state in said cell.

8-15. (canceled)
